(19) United States
(12) Patent Application Publication
(10) Pub. No.: US 2005/0033269 A1
(43) Pub. Date: Feb. 10, 2005

(54) Ferrule and enteral tube incorporating a ferrule

(75) Inventor: Christine Decaria, Salt Lake City, UT (US)

Correspondence Address: KIMBERLY-CLARK WORLDWIDE, INC., 401 NORTH LAKE STREET, NEENAH, WI, 54956, US

(73) Assignee: Kimberly-Clark Worldwide, Inc.

(21) Appl. No.: 10/635,729

(22) Filed: Aug. 6, 2003

(51) Int. Cl. A61M 25/16

(52) U.S. Cl. 604/533

(57) ABSTRACT

A ferrule is provided which comprises an inner wall defining a conduit extending between an inlet opening and an outlet opening; a first surface is defined by a first region of the inner wall; and a second surface is defined by a second region of the inner wall.

### BACKGROUND OF THE INVENTION

Enteral tubes for providing food and medication to a patient are well known. For example, U.S. Pat. No. 4,666,433, entitled Gastrostomy Feeding Device, invented by Parks and issued May 19, 1987; U.S. Pat. No. 4,701,163, entitled Gastrostomy Feeding Device, invented by Parks and issued Oct. 20, 1987; U.S. Pat. No. 4,798,592, entitled Gastrostomy Feeding Device, invented by Parks and issued Jan. 17, 1989; and U.S. Pat. No. 4,685,901, entitled Gastro-Jejunal Feeding Device, invented by Parks and issued Aug. 11, 1987 disclose earlier feeding tubes.

Referring to the illustrative drawing of FIG. 1A, there is shown a perspective view of an earlier enteral feeding device 20. The device 20 includes an elongated tubular member 51 formed from a stretchable elastomeric material such as silicone. FIG. 1B is an illustrative cross-sectional view of the tubular member 51 of the earlier device. The tubular member 51 defines a jejunal tube 22, a gastronomy tube 34 and a fluid line 46.

The jejunal feeding tube 22 (FIGS. 1A and 1B) includes an outlet end portion 24 (FIG. 1A) which can extend through a patient's stomach into the jejunum. The jejunal tube outlet end portion includes perforations 26 (FIG. 1A) which permit liquid food or medication to pass therethrough. The tube 22 (FIGS. 1A and 1B) is integrally connected to a jejunal tube inlet end portion 28 (FIG. 1A) which defines a jejunal inlet port 30 (FIG. 1A) having a removable plug cover 32 (FIG. 1A).

The gastrostomy tube 34 (FIG. 1A) is shorter than the jejunal tube 22 (FIGS. 1A and 1B) and includes a plurality of drainage inlets or food outlet ports such as inlet/outlet 36 (FIG. 1A). A gastrostomy tube end portion 37 (FIG. 1A) defines a gastrostomy inlet port 38 (FIG. 1A) having a plug cover 40 (FIG. 1A).

An inflatable balloon 42 is provided near the end of the gastrostomy tube 34 (FIGS. 1A and 1B) and is inflatable through a valve 44. The valve 44 is used to supply fluid to the balloon 42 through the fluid line 46 (FIGS. 1A and 1B).

Frictional contact between the elongated tubular member 51 (FIGS. 1A and 1B) and a locking ring 56 (FIG. 1A) is sufficiently great to prevent the tubular member 51 (FIGS. 1A and 1B) from moving further into the stomach. The locking ring 56 (FIG. 1A) remains in contact with a patient's abdominal wall during use. However, the frictional contact is sufficiently low to permit adjustment of placement of the tubular member 51 (FIGS. 1 and 1B) relative to a patient's abdomen.

Referring to the illustrative drawings of FIG. 2, there is shown a perspective view of an earlier device 20 in use. The inflated balloon 42 forms a gasket that seals the entrance to the stomach, and together with the locking ring 56, secures the device 20 in place.

While prior feeding tubes generally have been acceptable, there have been shortcomings with their use. In particular, for example, in order to provide food or medication to the jejunal inlet port 30 (FIG. 1A) of device 20 (FIG. 1A), a connector, such as a first connector 58 illustrated in FIG. 3 or a second connector 60 illustrated in FIG. 4, is inserted through the jejunal inlet port 30 (FIG. 1A). The inserted connector 58 (FIG. 3) or 60 (FIG. 4) is mechanically coupled to the jejunal inlet port 30 (FIG. 1A) and serves as a conduit between the jejunal tube 22 (FIGS. 1A and 1B) and an external feeding tube 62 or 64, shown in FIGS. 3 and 4 respectively. The external tube 62 or 64 is connected to a source of food such as a feeding bag (not shown).

In practice, connectors 58 or 60 such as those shown in FIGS. 3 and 4, for example, may be inserted into and removed from the jejunal inlet port 30 (FIG. 1A) or the gastrostomy inlet port 38 (FIG. 1A) numerous times during the course of use of the device 20 (FIGS. 1A and 2) which can be installed in a patient's stomach for extended periods of time. As mentioned above, the tubular member 51 (FIGS. 1A and 1B) which defines the jejunal tube inlet end 28 (FIG. 1A), and the gastrostomy tube end portion 37 (FIG. 1A) can be formed from a stretchable elastomeric material such as silicone. In order to produce an adequate mechanical coupling between the connector 58 (FIG. 3) or 60 (FIG. 4) and either the jejunal inlet port 30 (FIG. 1A) or the gastrostomy inlet port 38 (FIG. 1A), the connector is forced into place so as to produce a frictional engagement. Repeated insertions and removals of such connectors 58 (FIG. 3) or 60 (FIG. 4) can cause the jejunal inlet port 30 (FIG. 1A) or the gastrostomy inlet port 38 (FIG. 1A) to become somewhat stretched and deformed over time.

Unfortunately, as the jejunal and gastrostomy inlet ports 30, 38 (FIG. 1A) become more and more stretched in this manner, the tendency of a medical attendant responsible for coupling such a connector to the inlet ports 30, 38 often is to more forcibly push the connector into the jejunal or gastrostomy ports 30 or 38 resulting in still further stretching. Moreover, more force often must be exerted to dislodge a connector after such a forced insertion. Additionally, as the interior of the inlet ports 30, 38 becomes soiled with food oils, for example, an attendant may attempt to push a connector into the port even more forcibly in order to compensate for the slipperiness of such oils, causing further deformation of the port opening.

The problem of achieving a tight fit between a jejunal or gastrostomy inlet port 30 or 38 (FIG. 1A) and such connectors 58 (FIG. 3) or 60 (FIG. 4), for example, has been exacerbated by the fact that in the past, such connectors often have been available in a variety of shapes and sizes. This variety will be apparent from the illustrative drawings of FIGS. 3 and 4 in which the first and second connectors 58, 60 have quite different shapes. Consequently, in the past it often has been desirable to construct jejunal or gastrostomy inlet ports, that can accommodate any of a variety of such differently shaped connectors. Unfortunately, such earlier inlet ports often could not readily accommodate such a variety of differently shaped connectors without the need to forcibly insert or forcibly remove the connectors.

Even with the advent of feeding tubes incorporating ferrules, the variety of connectors which are frequently used therewith can still lead to the forcing of the connector and the feeding tube together to make a secure connection. Depending on the tube and connector being used this forcing still may not be sufficient to create a connection which retains the connector in the tube so as to avoid unintentional and/or undesired disconnection. Further, if sufficient force is applied during the connection of the prior connectors and tubes it may be such that the components are difficult to separate when desired. Such difficulties in separation may result in displacement of the feeding tube and/or discomfort to the patient during the attempted separation or even after the connector is ultimately separated from the tube.

Thus, there has been a need for a device to permit any of a variety of different shapes and sizes of connectors to be inserted into or removed from an inlet port of a feeding tube without the need to use excessive force and substantially without deforming the feeding tube inlet port while still providing for the retention of the connector. The present invention meets these needs.

### SUMMARY OF THE INVENTION

In response to the difficulties and problems discussed above, a ferrule adapted to be releasably interlocked with a connector has been developed. The devices contemplated by the present invention are configured to receive a connector having a protrusion thereon.

More specifically, one aspect of the present invention is directed to a ferrule adapted for use in a feeding tube formed from flexible material. The ferrule generally includes a wall formed from a hard substantially non-deformable material which defines a conduit extending along a central axis between an inlet opening and an outlet opening. The wall has a slot which is configured to receive a protrusion of a connector and releasably interlock the ferrule and connector. The ferrule also has a first surface defined by a first region of the wall which is inclined relative to the central axis so as to be dimensioned as a first luer and a second surface defined by a second region of the wall which is inclined relative to the central axis so as to be dimensioned as a second luer, wherein the first surface and the second surface are adapted for engagement with a portion of the connector.

Another aspect of the present invention is directed to a ferrule including a wall which defines a conduit extending along a central axis between an inlet opening and an outlet opening. The wall has a slot configured to receive a protrusion of a connector and releasably interlock the ferrule and a connector. A first surface of the ferrule is defined by a first region of the wall which is inclined relative to the central axis so as to be dimensioned as a first luer. A second surface of the ferrule is defined by a second region of the wall which is inclined relative to the central axis so as to be dimensioned as a second luer. The first region of the wall defines a first segment of the conduit which has a diameter that decreases with increasing distance from the inlet opening and the second region of the wall defines a second segment of the conduit which has a diameter that decreases with increasing distance from the inlet opening. The first surface and the second surface may be adapted for engagement with a portion of the connector. The wall may be formed from a hard substantially non-deformable material.

Yet another aspect of the present invention is directed to a ferrule adapted for use in a feeding tube. The ferrule includes a wall that has at least one slot and is formed from a hard substantially non-deformable material which defines a conduit extending along a central axis between an inlet opening and an outlet opening. Each slot of the ferrule has at least two portions or legs and each of the slots are configured to receive a protrusion of a connector and releasably interlock the ferrule and connector.

One aspect of the present invention is also directed to a feeding device having an elongated tube formed from a flexible material and a ferrule. The elongated tube has an inlet end portion formed from the flexible material and integrally connected to the elongated tube and defining an inlet port opening. The ferrule is disposed within the inlet end portion. The ferrule has a wall formed from a hard substantially non-deformable material which defines a conduit extending along a central axis between an inlet opening and an outlet opening. The wall has a slot that is configured to receive a protrusion of a connector and releasably interlock the ferrule and the connector. Within the ferrule, a first surface is defined by a first region of the wall which is inclined relative to the central axis so as to be dimensioned as a first luer and a second surface is defined by a second region of the wall which is inclined relative to the central axis so as to be dimensioned as a second luer such that the first surface and the second surface are adapted for engagement with a portion of the connector.

Still another aspect of the present invention relates to a feeding device having an elongated tube formed from a flexible material and a ferrule. The elongated tube has an inlet end portion formed from the flexible material and integrally connected to the elongated tube and defining an inlet port opening. The ferrule is disposed within the inlet end portion of the tube and has a wall formed from a hard substantially non-deformable material which defines a conduit extending along a central axis between an inlet opening and an outlet opening. The wall of the ferrule has at least one slot configured to receive a protrusion of a connector and releasably interlock the ferrule and the connector. Each of the slots has at least two portions or legs in which a protrusion may fit.

The invention will be more fully understood and further features and advantages will become apparent when reference is made to the following detailed description of exemplary embodiments of the invention and the accompanying drawings.

### BRIEF DESCRIPTION OF THE DRAWINGS

The purpose and advantages of the present invention will be apparent to those skilled in the art from the following detailed description in conjunction with the appended drawings in which:

FIG. 1A is a perspective view of an earlier feeding tube;

FIG. 1B is a cross-sectional view along line 1B-1B of FIG. 1A;

FIG. 2 is a perspective partially cutaway view of an earlier feeding tube installed in a patient;

FIGS. 3 and 4 are side elevation views of earlier connectors for insertion into end portions of a feeding tube;

FIG. 5 is a perspective view of a ferrule in accordance with the present invention;

FIG. 5A is a perspective view of an alternate ferrule in accordance with the present invention;

FIGS. 6A and 6B are top and bottom elevation views of the ferrule of FIG. 5;

FIG. 7 is a cross-sectional side elevation view of the ferrule of FIG. 5A;

FIG. 7A is a cross-sectional side elevation view of the ferrule of FIG. 5A;

FIG. 8 is a cross-sectional side elevation view of a ferrule incorporated into an end portion of a feeding tube;

FIG. 9 is an alternative embodiment of a ferrule in accordance with the invention;

FIG. 10 is a cross-section side elevation view of a ferrule in accordance with the invention;

FIG. 11 is a perspective view of a connector having a protrusion; and

FIG. 12 is a side elevational view of a ferrule in accordance with the invention.

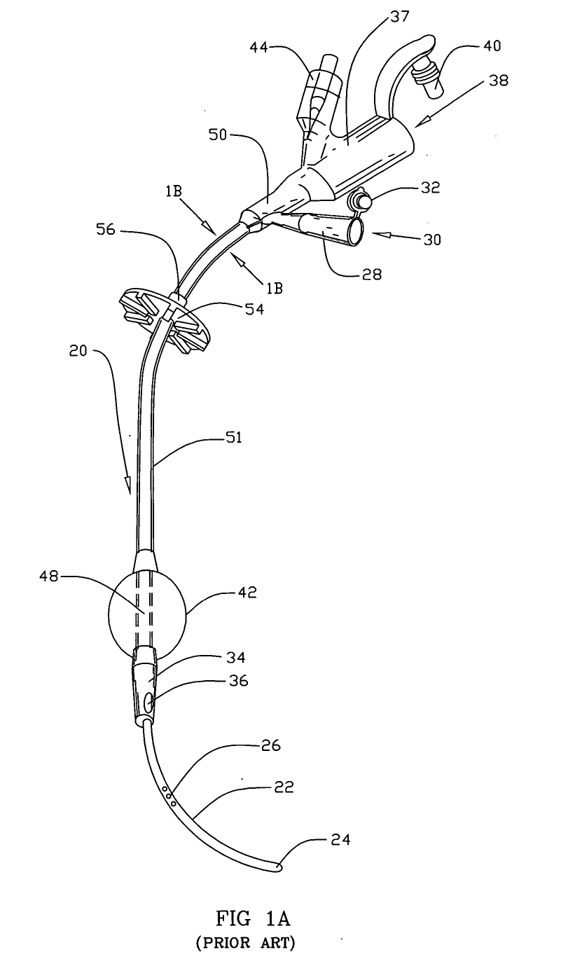

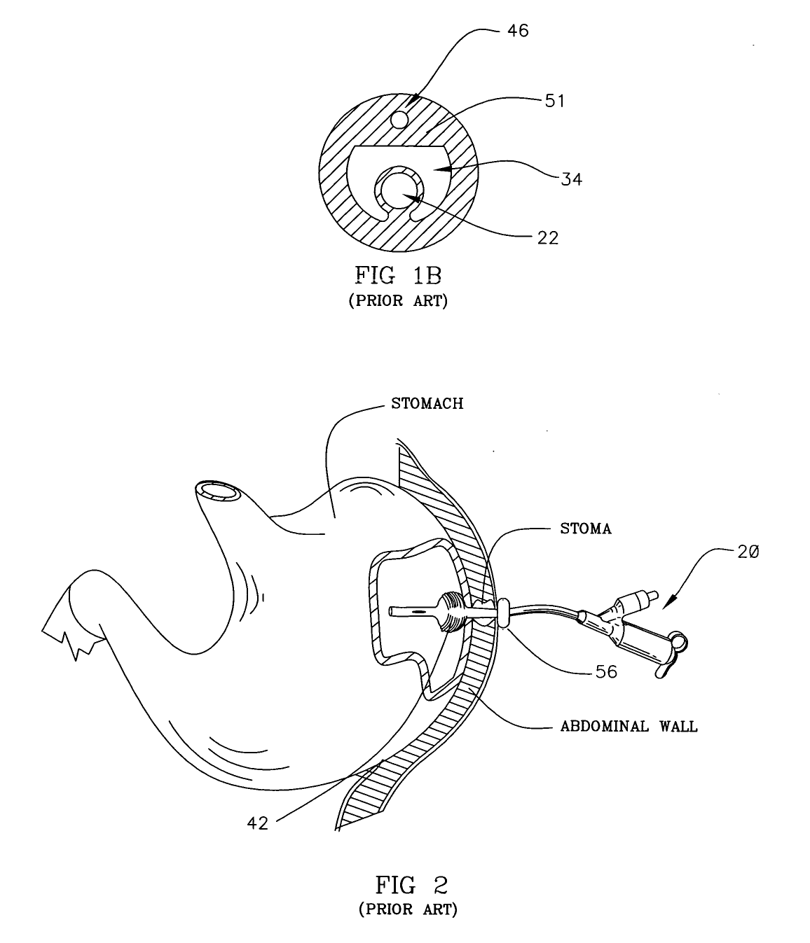

### DETAILED DESCRIPTION OF THE PREFERRED EMBODIMENTS

The following detailed description will be made in the context of a ferrule which is adapted for medical use. It is readily apparent, however, that the article of the present invention would also be suitable for use with other types of systems, circuits or conduits and the like and is not intended to be limited to medical devices or use in a medical field. In addition, the invention will be described in the context of its various configurations. It should be appreciated that alternative arrangements of the invention can comprise any combination of such configurations. As such, the use of a desired embodiment, a ferrule adapted for use with a feeding tube, for ease in understanding and describing the invention shall not, in any manner, limit the scope of the invention.

Reference will now be made to the drawings in which the various elements of the present invention will be given numeral designations and in which the invention will be discussed so as to enable one skilled in the art to make and use the invention. It should be appreciated that each example is provided by way of explaining the invention, and not as a limitation of the invention. For example, features illustrated or described with respect to one embodiment may be used with another embodiment to yield still a further embodiment. These and other modifications and variations are within the scope and spirit of the invention.

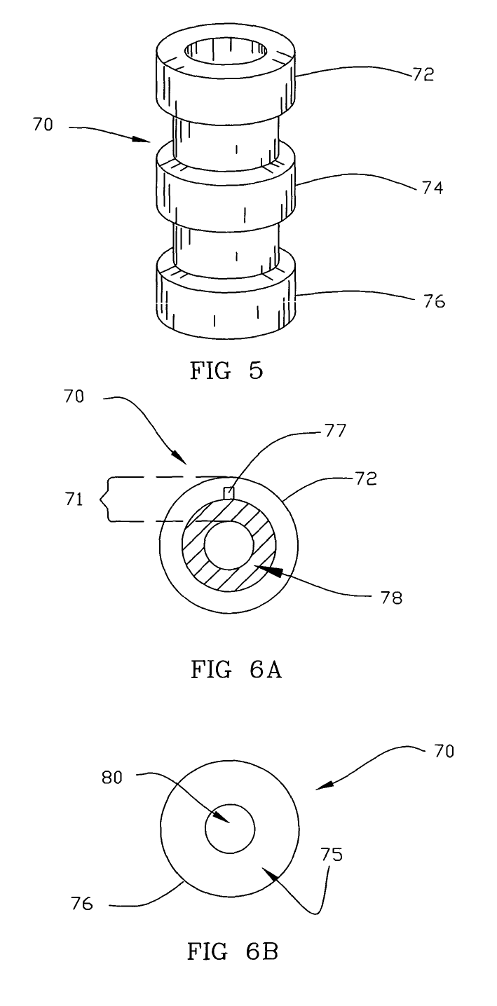

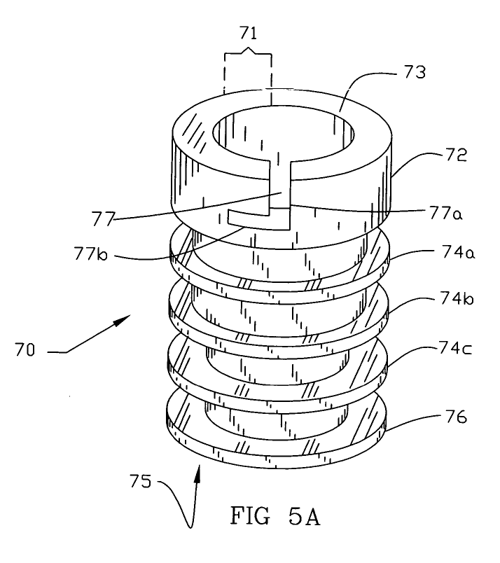

Turning now to the drawings, and FIG. 5 in particular, there is illustrated a perspective view of a ferrule 70 in accordance with a present embodiment of the invention. The ferrule 70 is generally cylindrical in shape and is shown with top, middle and bottom annular ribs 72, 74 and 76 extending outwardly therefrom. Although not shown in FIG. 5, the ferrule 70 has a slot which extends across or through a portion of wall 71 (FIG. 6A) of the ferrule 70.

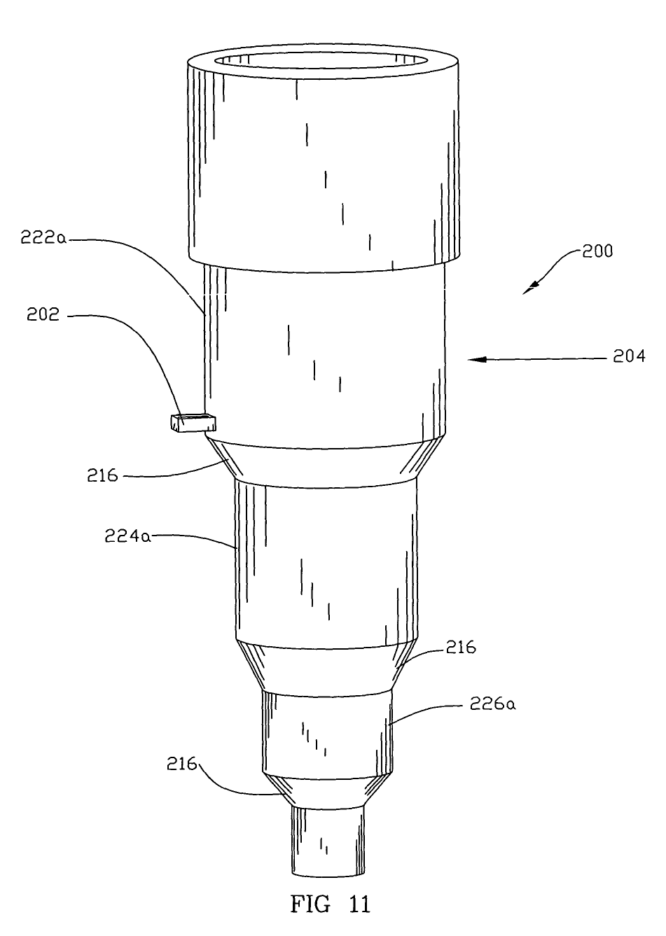

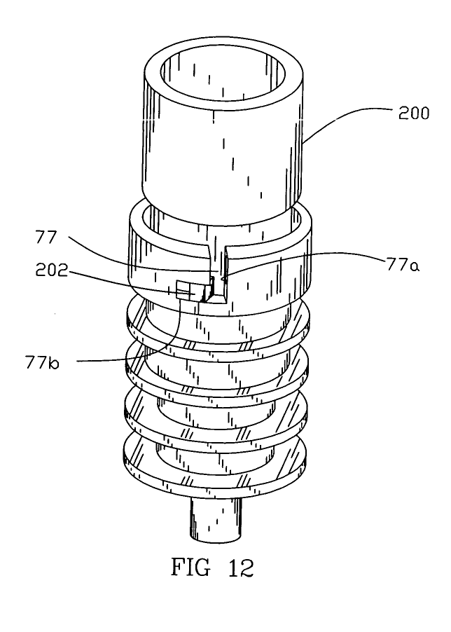

FIG. 5A illustrates another exemplary configuration of a ferrule 70 of the present invention. As shown, this embodiment includes a slot 77 which extends through the wall 71 of the ferrule. As seen in FIG. 5A, the number of ribs 72, 74a-c, and 76 may vary from embodiment to embodiment. As illustrated the slot 77 has a first portion or leg 77a and a second portion or leg 77b. The slot 77 is adapted to receive a protrusion 202 (FIGS. 11 and 12) on a connector 200 (FIGS. 11 and 12) inserted into the ferrule 70. It is contemplated that the slot 77 may extend through entire width of the wall 71 of the ferrule 70 as in FIG. 5A, however, it is also contemplated that the slot may only extend through a portion of the wall 71 of the ferrule 70 (e.g. as to form a recess in the wall) as in the ferrule shown in FIG. 6A. Also, while shown between end surface 73 and rib 74a in FIG. 5A, slot 77 may be in any suitable position in ferrule 70. That is, for example, at least a portion of the slot 77 may extend through at least a portion of the end surface 73 (FIG. 5A) of the ferrule; however, depending on the size of the connector and the location of the protrusion 202 (FIGS. 11 and 12) thereon, the slot 77 (FIGS. 5A and 12) in the ferrule 70 (FIGS. 5-8) may be located closer to the end 75 (FIGS. 5A and 6B) of the ferrule opposite end surface 73, and the slot need not extend into or through end surface 73. Depending on the size of the slot 77 (FIGS. 5A and 12) and the extent to which it extends into or through the wall 71 (FIGS. 5A and 6A) of the ferrule 70 (FIGS. 5, 5A and 12), a suitable connector may be selected. Alternatively, a suitable second member may be selected based on the dimensions of a connector that is selected for use.

Further, although not illustrated, more than one slot 77 (FIGS. 5A, 6A, 7 and 12) may be present in a ferrule 70 (FIGS. 5, 5A, 7 and 12). Multiple slots 77 in a ferrule 70 would provide the opportunity to use the ferrule 70 with a connector having multiple protrusions (not shown) and/or the ability to be used with connectors having different protrusion configurations either of which may lead to the creation of a better seal between the components and/or better retention of the connector. Furthermore, a portion of the slot 77 (FIGS. 5A, 6A, 7 and 12), desirably the second portion 77b (FIGS. 5A and 12), may be tapered (not shown) at least in part so as to be configured to create a friction fit with the protrusion 202 (FIGS. 11 and 12) of the connector 200 (FIGS. 11 and 12) inserted into the ferrule 70.

FIGS. 6A and 6B respectively show top elevation and bottom elevation views of a ferrule 70. In FIG. 6A, there is shown an inlet opening 78 generally surrounded by the top annular rib 72. In FIG. 6B, there is shown an outlet opening 80 surrounded by the bottom annular rib 76. In one embodiment, the ferrule 70 may be formed from a hard substantially non-deformable material such as plastic, metal, glass or polyvinylchloride. Desirably, the ferrule 70 is formed from a material that is acid-resistant and gamma-stabilized so that it can withstand a sterilization process involving irradiation.

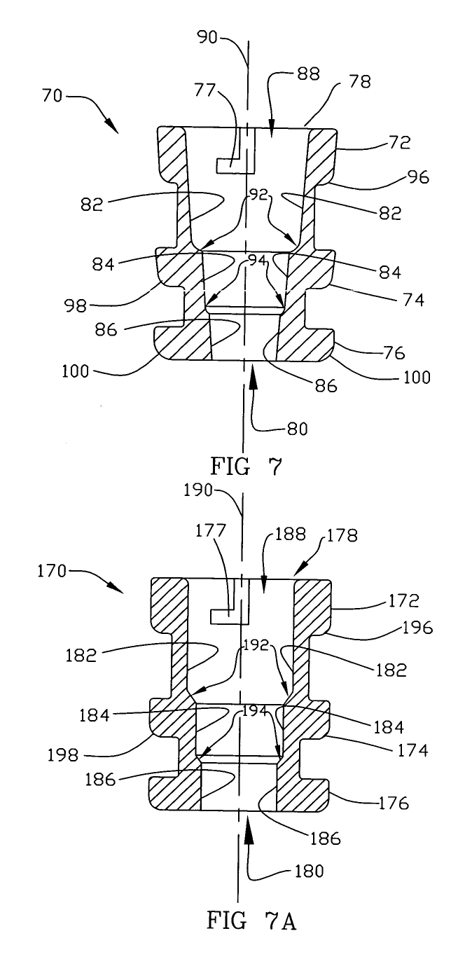

The illustrative drawing of FIG. 7, shows a cross-sectional view of the ferrule 70 of FIG. 5A, with the slot 77 visible in FIG. 7. First, second and third interior wall regions exemplarily shown as 82, 84 and 86 define a conduit 88 extending between the inlet opening 78 and the outlet opening 80. The respective first, second and third interior wall regions 82, 84 and 86 are each shown having substantially cylindrical contours and are aligned along a central axis 90 of the conduit 88.

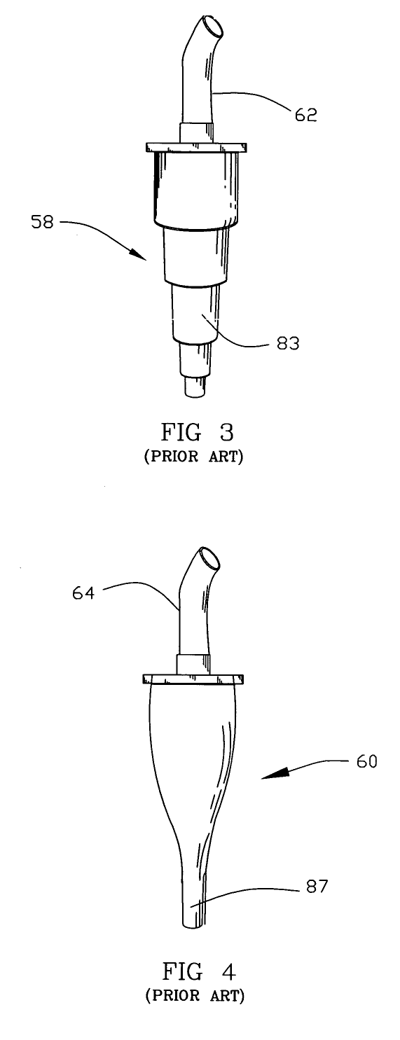

As explained more fully below, the first, second and third interior wall regions 82, 84 and 86 may define three separate surfaces which may be taper lock surfaces. Each of these interior wall regions can be sized and contoured to conform to the shape of a different portion of a connector or to multiple connectors so as to enable the use of ferrule 70 with a variety of connectors. For example, the first interior wall region 82 can be sized and contoured to conform to the shape of a portion of a connector such as that shown as 58 in FIG. 3 or 60 in FIG. 4 or 200 in FIG. 12. The second interior wall region 84 (FIG. 7), for example, can be sized and contoured to conform to the shape of another portion of the connectors in FIGS. 3, 4, or 12 or another connector altogether. Moreover, the third interior wall region 86 (FIG. 7), for example, can be sized and contoured to conform to the shape of yet another connector (not shown) with a narrower body shape or another portion of the connectors discussed above. It will be appreciated that the interior wall regions of FIG. 7 are drawn to a different scale than the connectors of FIGS. 3-4, 11 and 12.

A taper lock may be caused by a frictional engagement force that results when a connector 200 (FIGS. 11 -12) becomes lodged within the ferrule 70 (FIGS. 5-8) because of, for example, tapered surfaces 82, 84, 86, 92, 94 (FIG. 7) or 216, 222a, 224a, 226a (FIGS. 11-12) which come in contact with another surface which is also desirably tapered. Different embodiments of the present invention may exhibit different sizing and contouring of the different interior wall regions (such as 82, 84 and 86 in FIG. 7) so as to provide one or more different interior wall regions which can be lodged against differently sized and contoured portions of a connector.

Thus, for example, when a connector such as that shown as 200 in FIGS. 11 and 12 is inserted into the inlet opening 78 (FIGS. 6A and 7) of the ferrule 70 (FIGS. 5-8), it can become lodged against the first interior wall region 82 (FIG. 7), desirably forming a taper lock with it. Alternatively, for example, connector 58 (FIG. 3) could become lodged against a shoulder of the ferrule such as 92 or 94 (FIG. 7). Likewise, when a connector having appropriate dimensions is inserted into the inlet opening 78 of the ferrule 70, it can become lodged against one or more of the other interior wall regions (e.g. 84 or 86), forming a taper lock or the like therewith. In accordance with the present invention, it will be appreciated that the creation of a taper lock or the like between the ferrule 70 and a connector desirably should not prevent the rotation or movement of connector relative to the ferrule 70 so as to enable the interlocking discussed herein. As discussed below, the present invention also contemplates instances in which a taper lock or other seal between the ferrule 70 and one or more wall regions of a connector is not made.

A more detailed description of the desired sizing and contour of one embodiment of the ferrule 70 of FIG. 7 follows. The first interior wall region 82 is shown with walls 82, 84 and 86 that are inclined relative to the central axis 90 so as to define a generally conical shape in which a diameter of a first segment of the conduit 88 defined by the first interior wall region 82 decreases with increasing distance from the inlet opening 78. A first interior annular shoulder 92 demarcates the end of the first interior wall region 82 of the ferrule of FIG. 7.

As will be appreciated, the interior walls may engage one or more of the regions of the body 204 (FIGS. 11 and 12) of a connector 200 (FIGS. 11 and 12) of the present invention so as to assist in the connection and/or retention of the components relative to each other and/or to reduce or minimize fluid leaks between the components.

A second segment of the conduit 88 is shown in FIG. 7 as being defined by the second interior wall region 84 which also is substantially conical in shape. Like the first interior wall region 82, the walls of the second interior wall region 84 may be inclined relative to the central axis 90 such that the diameter of a second conduit segment decreases with increasing distance from the inlet opening 78. A second interior annular shoulder 94 demarcates the end of the second interior wall region 84 of the ferule of FIG. 7.

A third segment of the conduit 88 is shown in FIG. 7 as being defined by the third conical interior wall region 86. The walls of the third interior wall region 86 are shown inclined relative to the central axis 90 such that the diameter of the third conduit segment decreases with increasing distance from the inlet opening 78. In one embodiment, the dimensions of one or more of the wall regions, e.g., 82, 84, 86 may be those of a luer so as to be adopted to engage a portion of a connector.

It will be understood that alternative ferrules can, for example, be constructed in accordance with the invention in which the wall regions are generally parallel to central axis, but that a portion of the wall region is inclined relative to the central axis of the ferrule so as to be dimensioned as luer. For example, in FIG. 7A, wall regions 182, 184 and 186 of ferrule 170 are shown as being generally parallel to central axis 190, while surfaces 192 and 194 are shown as being inclined relative to the central axis 190 and may be dimensioned as luers.

Additionally, one will appreciate that, although the above described embodiments disclose smooth inner wall regions (e.g., 82, 84, 86 shown in FIG. 7), a taper-lock can be formed in which inner wall regions have contours formed in them such as ridges, steps, bumps and the like. Alternatively, the connector may be formed such that the outer surfaces (e.g. 214 in FIG. 5) have contours formed in them such as ridges, steps, bumps and the like.

Referring again to FIGS. 5, 5A, 6A and 7, the outer edges of the middle and bottom outwardly extending annular ribs 74 (FIGS. 5 and 7), 76 (FIGS. 5A, 6A and 7) may be and are shown inclined relative to the central axis 90 (FIG. 7) such that the diameter of each of these respective annular ribs 74, 76 decreases with increasing distance from the inlet opening 78 (FIGS. 6A and 7). Moreover, the respective shoulders 96, 98 and 100 (FIG. 7) of the top, middle and bottom annular rings are shown as being rounded. The inclined and rounded edges of the outwardly protruding annular ribs can facilitate the process of inserting the ferrule 70 (FIGS. 5 and 7) into an inlet end portion of a feeding tube as explained more fully below.

As noted and discussed in more detail herein, a connector 200 (FIGS. 11 and 12) and ferrule 70 (FIGS. 5-8) may create a lock or seal (e.g. a taper-lock) between one another; however, as discussed in more detail below, the ferrule 70 also has at least one slot 77 (FIGS. 5A and 7) therein which is capable of receiving a protrusion 202 (FIGS. 11 and 12) on a connector 200 such as that shown in FIGS. 11 and 12 to assist in retaining or in maintaining the position of the ferrule and the connector when inserted therein. It will be appreciated that while the term slot is used throughout this disclosure for ease of reading and understanding, and while not intending to be limited thereby, the term slot is intended to also include, groove, channel, opening, recess, aperture and the like. It will also be appreciated that while reference is made to a protrusion 202 (FIGS. 11 and 12), any suitable guide, flange, extension, prong, or the like also may be used. However, for ease of reading and understanding of this disclosure, and not intending to be limited thereby, protrusion will be used hereafter.

Although illustrated in FIG. 5A in a generally “L” shape, slot 77 of the ferrule 70 may be of any suitable shape and size. That is, for example, the second portion 77b of an “L-shaped slot” may be inclined or declined relative to the central axis 90 (FIG. 7) of the ferrule 70 or the slot may be at least partially curved, T-shaped, U-shaped or the like. It is also contemplated that the slot 77 may have in at one least portion thereof a non-smooth surface or other mechanism (not shown) so as to enhance the ability of the ferrule 70 (FIGS. 5, 5A and 12) to retain its position with a connector 200 (FIGS. 11 and 12) so as to minimize or reduce inadvertent or undesired separation of the components.

Although desired that the ferrule of the present invention be used with a connector having a protrusion, it is contemplated that a ferrule 70 (FIGS. 5-8) with a slot 77 (FIG. 5A) may be used in conjunction with either a connector 200 having a protrusion 202 (FIGS. 11 and 12) as discussed above or with a connector without protrusion such as those shown in FIGS. 3 and 4. When the ferrule 70 of the present invention is used in conjunction with a connector having a protrusion which is capable of fitting within slot 77 (FIG. 5A) and acting in conjunction with the slot 77 to retain or assist in maintaining the positioning of the connector 200 (FIGS. 11 and 12) relative to the ferrule 70 (FIGS. 5-8), the protrusion 202 (FIGS. 11 and 12) should be aligned with the slot 77 (FIG. 5A) to allow the connector 200 (FIGS. 11 and 12) to be inserted into the ferrule 70 as illustrated in FIG. 12. The ferrule 70 (FIGS. 5-8 and 12), and more specifically the slot 77 (FIGS. 5A and 12) therein, is desirably designed such that a protrusion 202 (FIGS. 11 and 12) may be closely received first within a first portion or leg 77a (FIGS. 5A and 12) of slot 77 (FIGS. 5A and 12). Then, depending on the embodiment, the connector 200 (FIGS. 11 and 12) and protrusion 202 (FIGS. 11 and 12) may be rotated relative to the ferrule 70 (FIGS. 5-8 and 12) so as to be received within the intersecting second portion or leg 77b (FIGS. 5A and 12) of slot 77 (FIGS. 5A and 12) in a manner to releasably interlock the connector 200 (FIGS. 11 and 12) to the ferrule 70 (FIGS. 5-8 and 12). Such an interlocking connection can provide a way to retain the position of a connector relative to a ferrule, even in those instances where a taper lock or other connection or seal between the outer surface of the connector and the ferrule (as discussed above) is not present or achieved.

It will be appreciated that in any embodiment, but especially those where a taper lock or the like is not created between the connector and the ferrule, the ferrule may include a sealing member or the like (not shown) desirably made of an elastomeric material or the like, which is capable of allowing at least a portion of a connector to pass there through yet still create or form a seal, gasketing effect or the like about the connector when inserted in the ferrule such that fluid leaks from the ferrule are eliminated or reduced. It will be appreciated that the sealing member may be located between the end surface 73 (FIG. 5A) and the outlet opening 80 (FIGS. 6B and 7) of the ferrule 70 (FIGS. 5-8), but is desirably located closer to the end surface 73 of the ferrule 70. Exemplary material for the sealing member includes, for example, an elastomeric material or the like and an exemplary embodiment may take the form of an o-ring, gasket, or the like. Such a seal or the like can act to reduce, minimize or prevent leakage from the ferrule when the connector is inserted therein.

In the embodiment shown in FIG. 12, when it is desired to disconnect the connector 200 from the ferrule 70, the connector 200 and ferrule 70 should be rotated relative to one another such that the protrusion 202 is aligned with first portion or leg 77a of slot 77 of the ferrule 70. Once the protrusion is aligned with the leg 77a of slot 77, the connector 200 may be withdrawn from the ferrule 70 when sufficient force in the proper direction is applied.

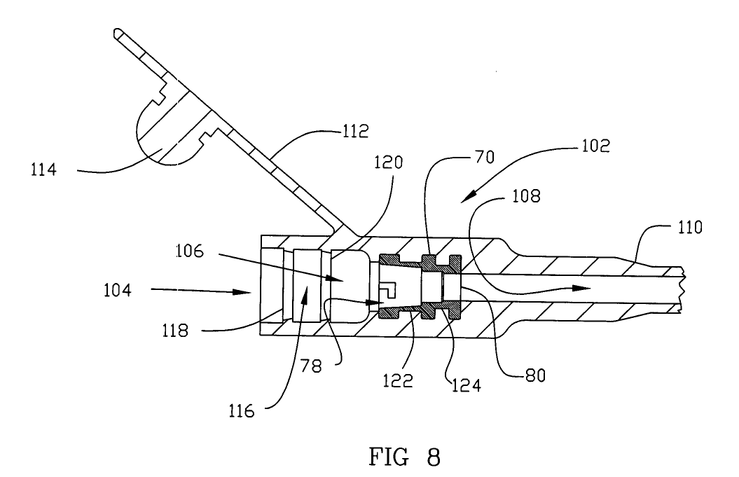

Referring now to FIG. 8, there is shown a cross-sectional elevation view of an inlet end portion 102 of an enteral tube, in accordance with the embodiment of the invention, which incorporates the ferrule 70. The feeding tube is generally formed from an elastomeric silicone material and can be formed by injection molding. The inlet end portion 102 defines an inlet port opening 104 and defines a first passage 106 between the inlet port opening 104 and the inlet opening 78 of the ferrule 70. The outlet opening 80 of the ferrule 70 communicates with a second elongated passage 108 defined by an elongated tube portion 110 of the feeding tube, only a short segment of which is shown.

An arm 112 is shown integrally formed with the end portion 102 and has a plug 114 extending therefrom. The arm 112 can be bent, and the plug 114 can be inserted into the inlet port opening 104, whereupon it may become lodged in a space 116 between two inwardly projecting annular protrusions 118,120 integrally formed in the inlet end portion 102. In this manner, the opening 104 can be closed when the end portion 102 is not in use.

Moreover, when the inlet end portion 102 is in use and a connector (such as connectors 58, 60 and 200 shown in FIGS. 3, 4, 12, respectively) is inserted through the inlet port opening 104 and desirably has formed a taper lock or the like with one of the inner wall regions of the ferrule 70, the inwardly projecting annular protrusions 118, 120 may abut against the connector. Desirably, the protrusions 118, 120 advantageously produce a fluid seal with a connector inserted through the inlet port opening 104 to prevent fluid leakage from the opening 104. Although the presence of protrusions 118, 120 is not required in the present invention, it will be appreciated that such annular protrusions alternatively could be inlaid within an annular inset (not shown) or the like in the ferrule 70 depending on the location of the ferrule relative to the inlet port opening 104 to provide the same or similar benefits.

The outwardly projecting annular ribs 72, 74, 76 (FIGS. 5 and 5A) of the ferrule 70 (FIGS. 5-8) may be adapted to grip inwardly projecting annular ribs 122, 124 (FIG. 8) which are integrally formed in the end portion 102 (FIG. 8) and which are contoured to fit snugly between the ribs 72, 74, 76 (FIGS. 5 and 5A). In this manner, the ribs 72, 74, 76 (FIG. 5) hold the ferrule 70 (FIGS. 5-8) in place within the inlet end portion 102 (FIG. 8). It will be appreciated that although annular ribs 72, 74, 76 (FIGS. 5 and 5A) are used to grip the end portion 102 (FIG. 8), differently shaped objects could be used to accomplish that purpose. For example, the outer surface of the ferrule 70 (FIGS. 5-8) could be abraded so as to roughen it to allow it to grip the interior of the end portion 102 (FIG. 8). Alternatively, for example, the outer surface of the ferrule 70 (FIGS. 5-8) could have protrusions in the shape of individual upstanding barbs or in the shape of helical ridges.

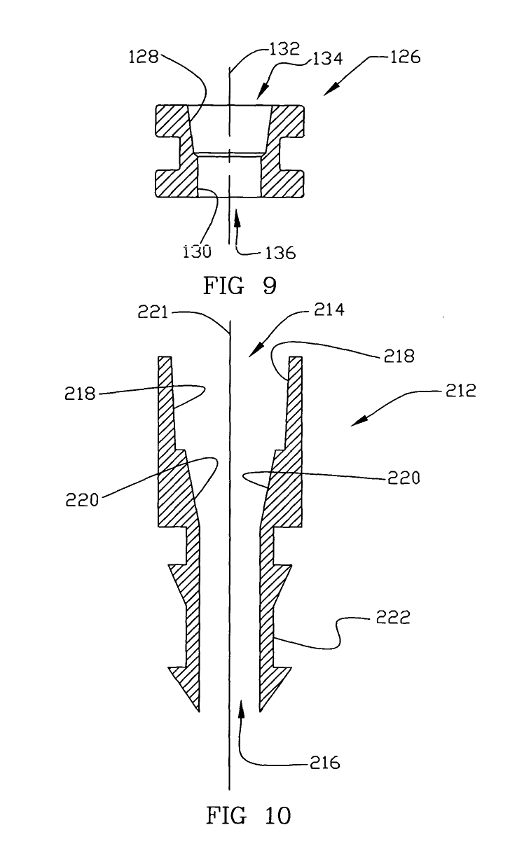

Referring to the illustrative drawing of FIG. 9, there is shown an alternative embodiment of a ferrule 126 in accordance with the invention. The alternative ferrule 126 is generally similar to the ferrule 70 (FIGS. 5-8) except that it includes only two interior wall regions 128, 130 instead of the three such regions 82, 84, 86 (FIG. 7) of the ferrule 70 (FIGS. 5-8). The ferrule 126 includes a conduit having a central axis 132. The conduit extends between an inlet opening 134 and an outlet opening 136. Thus, the structure and operation of the alternative ferrule 126 will be appreciated from the above description of ferrule 70 (FIGS. 5-8) and need not be set forth again herein. As above, it will be appreciated that a connector having a protrusion located at a point closer to the second end of the connector than in the embodiment shown in FIGS. 11 and 12 and/or a second protrusion (not shown) may be desirable and/or necessary for the connector and ferrule 126 to cooperatively engage each other.

In order to manufacture a feeding tube that incorporates a ferrule like that of FIGS. 7 or 9, the ferrule can be, for example, mounted on a pin, for example, and a silicone end portion can be injection molded about it. Alternatively, the silicone end portion can be produced first, and later the ferrule can be mounted on a mandril and be forced into position within the end portion. In this alternative manufacturing approach, inclined and rounded outer portions of the outwardly extending annular ribs are useful to ensure that the ferrule does not become snagged as it is forced into the end portion

It will be appreciated that while the embodiments of FIGS. 7 and 9 include interior wall regions 82, 84, 86 and 128,130, respectively, adapted for use in forming taper locks or the like with differently sized connector portions, the interior wall regions need not be smooth to be employed in forming a taper lock or other engagement with a connector or portion thereof.

Referring to the illustrative drawings of FIG. 10, there is shown a cross-sectional view of another ferrule 212 in accordance with the invention. The ferrule 212 defines a conduit extending between an inlet opening 214 and an outlet opening 216. It also defines first and second conical inner wall regions 218, 220, aligned about a central axis 221, that can be used to form respective taper locks with differently sized and contoured connector portions. A barbed stem 222 extends longitudinally along the axis and defines the outlet opening 216. The barbs can be used to secure the ferrule to an enteral tube inlet opening (not shown) so as to adapt such a tube opening to withstand repeated insertions and removals of differently sized connectors without suffering, stretching or wear-and-tear. Although not visible in FIG. 10, ferrule 212 also has a slot on at least one of the wall regions 218, 220 which is adapted to receive a protrusion on a connector for being used in conjunction with the ferrule.

Thus, the ferrules 70 (FIGS. 5-8), 126 (FIG. 9) and 212 (FIG. 10) in accordance with the present embodiments of the invention advantageously can be used to ensure that connectors such as connectors 58 (FIG. 3), 60 (FIG. 4), and 200 (FIG. 11) can be inserted and removed from an end portion of a feeding tube without the exertion of undue force and without resulting in unwanted stretching of the end portion. Furthermore, the inner wall regions of the ferrules 70 (FIG. 7), 126 (FIG. 9) and 212 (FIG. 10) can be sized and contoured to precisely conform to the shape of a number of different connector portions, however, as indicated above, the creation of a taper lock or the like between the connector and the ferrule while desired is not necessary. Further still, the ferrules of the present invention advantageously can reduce or minimize undesired disconnections or separations from a connector. Such disconnections or separations may be avoided even in those instances in which a taper lock or the like is not achieved as was required with previous ferrules and connectors.

While the invention has been described in detail with respect to specific embodiments thereof, those skilled in the art, upon obtaining an understanding of the invention, may readily conceive of alterations to, variations of, and equivalents to the described embodiments and the processes for making them. It is intended that the present invention include such modifications and variations as come within the scope of the appended claims and their equivalents.

1. A ferrule adapted for use in a feeding tube formed from flexible material comprising:
a wall formed from a hard substantially non-deformable material which defines a conduit extending along a central axis between an inlet opening and an outlet opening, the wall having a slot, wherein the slot is configured to receive a protrusion of a connector and releasably interlock the ferrule and connector;
a first surface defined by a first region of the wall which is inclined relative to the central axis so as to be dimensioned as a first luer; and
a second surface defined by a second region of the wall which is inclined relative to the central axis so as to be dimensioned as a second luer;
wherein the first surface and the second surface are adapted for engagement with a portion of the connector.

2. The ferrule of claim 1 further including a third surface defined by a third region of the wall which is inclined relative to the central axis so as to be dimensioned as a third luer, wherein the third surface is adapted for engagement with a portion of the connector.

3. The ferrule of claim 1 wherein the slot in the wall has a first portion and a second portion, the first portion of the slot generally being in parallel to the central axis, the second portion of the slot being intersecting with the first portion of the slot.

4. The ferrule of claim 3 wherein the second portion of the slot is tapered at least in part.

5. The ferrule of claim 1 wherein the wall has a width and the wherein the slot extends across the width of the wall.

6. The ferrule of claim 1 wherein the wall has a width and the wherein the slot extends across a portion of the width of the wall.

7. The ferrule of claim 1 wherein the slot is generally L-shaped.

8. The ferrule of claim 1 wherein the slot is generally T-shaped.

9. The ferrule of claim 1 wherein the ferrule has an end surface and wherein at least a portion of the slot extends through at least a portion of the end surface.

10. The ferrule of claim 1 wherein the first region of the wall defines a first segment of the conduit which has a diameter that decreases with increasing distance from the inlet opening.

11. The ferrule of claim 1 wherein the second region of the wall defines a second segment of the conduit which has a diameter that decreases with increasing distance from the inlet opening.

12. The ferrule of claim 1 wherein the first region of the wall forms a generally annular shape about the central axis, and the second region of the wall forms a generally annular shape about the central axis.

13. The ferrule of claim 1 wherein the first surface and the second surface are aligned along the central axis of the conduit; and the first surface is disposed closer to the inlet opening than the second surface.

14. The ferrule of claim 1 further comprises a gripping member for gripping the feeding tube.

15. The ferrule of claim 14 wherein the gripping member includes at least two outwardly extending annular ribs.

16. The ferrule of claim 1 further including a sealing member configured to assist in maintaining the position of a connector relative to the ferrule.

17. The ferrule of claim 1 wherein the sealing member is at least in part an elastomeric material.

18. A ferrule comprising:
a wall formed from a hard substantially nondeformable material which defines a conduit extending along a central axis between an inlet opening and an outlet opening, the wall having a slot, wherein the slot is configured to receive a protrusion of a connector and releasably interlock the ferrule and connector;
a first surface defined by a first region of the wall which is inclined relative to the central axis so as to be dimensioned as a first luer; and
a second surface defined by a second region of the wall which is inclined relative to the central axis so as to be dimensioned as a second luer;
wherein the first region of the wall defines a first segment of the conduit which has a diameter that decreases with increasing distance from the inlet opening;
wherein the second region of the wall defines a second segment of the conduit which has a diameter that decreases with increasing distance from the inlet opening; and
wherein the first surface and the second surface are adapted for engagement with a portion of the connector.

19. The ferrule of claim 18 further including a third surface defined by a third region of the wall which is inclined relative to the central axis so as to be dimensioned as a third luer; wherein the third region of the wall defines a third segment of the conduit which has a diameter that decreases with increasing distance from the inlet opening.

20. The ferrule of claim 18 wherein the first surface is a taper lock surface and wherein the second surface is a taper lock surface.

21. The ferrule of claim 18 wherein the first surface is disposed closer to the inlet opening than the second surface and the second surface is disposed closer to the inlet opening than the third surface.

22. The ferrule of claim 18 further including gripping member for gripping the feeding tube.

23. The ferrule of claim 22 wherein the gripping member includes at least two outwardly extending annular ribs.

24. A ferrule adapted for use in a feeding tube comprising:
a wall formed from a hard substantially non-deformable material which defines a conduit extending along a central axis between an inlet opening and an outlet opening, the wall having at least one slot, each of the slots having at least two portions;
the slot being configured to receive a protrusion of a connector and releasably interlock the ferrule and connector.

25. The ferrule of claim 24 wherein one of the portions of each of the slots is generally parallel to the central axis.

26. The ferrule of claim 24 wherein at least a portion of at least one segment of the conduit is inclined relative to the central axis so as to enable a friction fit with a portion of a connector.

27. The ferrule of claim 24 further including a sealing member wherein the sealing member is configured to assist in maintaining the position of a connector relative to the ferrule.

28. The ferrule of claim 24 wherein the sealing member is at least in part an elastomeric material.

29. A feeding device comprising:
an elongated tube formed from a flexible material;
an inlet end portion formed from the flexible material and integrally connected to the elongated tube and defining an inlet port opening; and
a ferrule disposed within the inlet end portion the ferrule including a wall formed from a hard substantially non-deformable material which defines a conduit extending along a central axis between an inlet opening and an outlet opening, the wall having a slot, wherein the slot is configured to receive a protrusion of a connector and releasably interlock the ferrule and connector;
a first surface defined by a first region of the wall which is inclined relative to the central axis so as to be dimensioned as a first luer; and
a second surface defined by a second region of the wall which is inclined relative to the central axis so as to be dimensioned as a second luer;
wherein the first surface and the second surface are adapted for engagement with a portion of the connector.

30. The feeding device of claim 29 wherein the ferrule further includes gripping member for gripping the inlet end portion.

31. The feeding device of claim 29 further including a third surface defined by a third region of the wall which is inclined relative to the central axis so as to be dimensioned as a third luer.

32. The feeding device of claim 29 wherein:
the first region of the wall defines a first segment of the conduit which has a diameter that decreases with increasing distance from the inlet opening; and
the second region of the wall defines a second segment of the conduit which has a diameter that decreases with increasing distance from the inlet opening.

33. The feeding device of claim 24 wherein the first region of the wall forms a generally annular shape about the central axis; and the second region of the wall forms a generally annular shape about the central axis.

34. A feeding device comprising:
an elongated tube formed from a flexible material;
an inlet end portion formed from the flexible material and integrally connected to the elongated tube and defining an inlet port opening; and
a ferrule disposed within the inlet end portion the ferrule including,
a wall formed from a hard substantially non-deformable material which defines a conduit extending along a central axis between an inlet opening and an outlet opening, the wall having at least one slot, each of the slots having at least two portions;
the slot being configured to receive a protrusion of a connector and releasably interlock the ferrule and connector.
